(19) United States
(12) Patent Application Publication
(10) Pub. No.: US 2011/0081316 A1
(43) Pub. Date: Apr. 7, 2011

(54) PYRAZOLE INHIBITORS OF PHOSPHATIDYLINOSITOL 3-KINASE

(75) Inventors: David Messersmith, Somerville, MA (US); Alex Aronov, Newton, MA (US); David J. Lauffer, Stow, MA (US); Anne-Laure Grillot, Somerville, MA (US); Robert J. Davies, Watertown, MA (US)

(73) Assignee: VERTEX PHARMACEUTICALS INCORPORATED, Cambridge, MA (US)

(21) Appl. No.: 12/896,042

(22) Filed: Oct. 1, 2010

Related U.S. Application Data
(60) Provisional application No. 61/248,013, filed on Oct. 2, 2009.

(51) Int. Cl. A61K 31/4439; C07D 401/06; C07D 401/14; C07D 403/10; A61K 31/498; A61K 31/4709; A61P 37/00; A61P 29/00; A61P 25/00; A61K 38/21; A61K 39/395; C12N 9/99

(52) U.S. Cl. 424/85.6; 546/275.4; 544/353; 546/175; 514/341; 514/249; 514/314; 424/133.1; 435/184

(57) ABSTRACT

The present invention relates to compounds useful as inhibitors of PI3K, particularly of PI3Kγ. The invention also provides pharmaceutically acceptable compositions comprising said compounds and methods of using the compositions in the treatment of various disease, conditions, or disorders.

### CROSS REFERENCE TO RELATED APPLICATIONS

This present application claims the benefit, under 35 U.S.C. §119, to U.S. Provisional Application No. 61/248,013, filed Oct. 2, 2009, the entire disclosures of which are incorporated herein by reference.

### TECHNICAL FIELD OF THE INVENTION

The present invention relates to compounds useful as inhibitors of phosphatidylinositol 3-kinase (PI3K). The invention also provides pharmaceutically acceptable compositions comprising the compounds of the invention and methods of using the compositions in the treatment of various disorders.

### BACKGROUND OF THE INVENTION

PI3Ks are a family of lipid kinases that catalyze the phosphorylation of the membrane lipid phosphatidylinositol (PI) on the 3′-OH of the inositol ring to produce PI 3-phosphate [PI(3)P, PIP], PI 3,4-bisphosphate [PI(3,4)P₂, PIP2] and PI 3,4,5-trisphosphate [PI(3,4,5)P₃, PIP3]. PI(3,4)P₂ and PI(3,4,5)P₃ act as recruitment sites for various intracellular signaling proteins, which in turn form signaling complexes to relay extracellular signals to the cytoplasmic face of the plasma membrane.

Eight mammalian PI3Ks have been identified so far, including four class I PI3Ks. Class Ia includes PI3Kα, PI3Kβ and PI3Kδ. All of the class Ia enzymes are heterodimeric complexes comprising a catalytic subunit (p110α, p110β or p110δ) associated with an SH2 domain-containing p85 adapter subunit. Class Ia PI3Ks are activated through tyrosine kinase signaling and are involved in cell proliferation and survival. PI3Kα and PI3Kβ have also been implicated in tumorigenesis in a variety of human cancers. Thus, pharmacological inhibitors of PI3Kα and PI3Kβ are useful for treating various types of cancer.

PI3Kγ, the only member of the Class Ib PI3Ks, consists of a catalytic subunit p110γ, which is associated with a p101 regulatory subunit. PI3Kγ is regulated by G protein-coupled receptors (GPCRs) via association with βγ subunits of heterotrimeric G proteins. PI3Kγ is expressed primarily in hematopoietic cells and cardiomyocytes and is involved in inflammation and mast cell function. Thus, pharmacological inhibitors of PI3Kγ are useful for treating a variety of inflammatory diseases, allergies and cardiovascular diseases.

Although a number of PI3K inhibitors have been developed, there is a need for additional compounds to inhibit PI3Ks for treating various disorders and diseases, especially those affecting the central nervous system (CNS). Accordingly, it would be desirable to develop additional compounds that are useful as inhibitors of PI3K that penetrate the blood-brain barrier (BBB).

### SUMMARY OF THE INVENTION

It has been found that compounds of this invention, and pharmaceutically acceptable compositions thereof, are effective as inhibitors of PI3K, particularly PI3Kγ. Accordingly, the invention features compounds having the general formula:

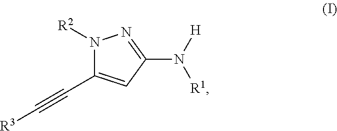

or a pharmaceutically acceptable salt thereof, where each of R¹, R², and R³ is as defined herein.

The invention also provides pharmaceutical compositions that include a compound of formula I and a pharmaceutically acceptable carrier, adjuvant, or vehicle. These compounds and pharmaceutical compositions are useful for treating or lessening the severity of a variety of disorders, including autoimmune diseases and inflammatory diseases of the CNS.

The compounds and compositions provided by this invention are also useful for the study of PI3K in biological and pathological phenomena; the study of intracellular signal transduction pathways mediated by such kinases; and the comparative evaluation of new kinase inhibitors.

### DETAILED DESCRIPTION OF THE INVENTION

### Definitions and General Terminology

As used herein, the following definitions shall apply unless otherwise indicated. For purposes of this invention, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted,” whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position.

As described herein, when the term “optionally substituted” precedes a list, said term refers to all of the subsequent substitutable groups in that list. For example, if X is halogen; optionally substituted C₁₋₃ alkyl or phenyl; X may be either optionally substituted alkyl or optionally substituted phenyl. Likewise, if the term “optionally substituted” follows a list, said term also refers to all of the substitutable groups in the prior list unless otherwise indicated. For example: if X is halogen, C₁₋₃ alkyl, or phenyl, wherein X is optionally substituted by J^{X}, then both C₁₋₃ alkyl and phenyl may be optionally substituted by J^{X}. As is apparent to one having ordinary skill in the art, groups such as H, halogen, NO₂, CN, NH₂, OH, or OCF₃ would not be included because they are not substitutable groups. If a substituent radical or structure is not identified or defined as “optionally substituted,” the substituent radical or structure is unsubstituted.

Combinations of substituents envisioned by this invention are preferably those that result in the formation of stable or chemically feasible compounds. The term “stable,” as used herein, refers to compounds that are not substantially altered when subjected to conditions to allow for their production, detection, and, preferably, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group,” as used herein, means a straight-chain (i.e., unbranched) or branched, substituted or unsubstituted hydrocarbon chain that is completely saturated or that contains one or more units of unsaturation. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments, aliphatic groups contain 1-4 carbon atoms. Suitable aliphatic groups include, but are not limited to, linear or branched, substituted or unsubstituted alkyl, alkenyl, or alkynyl groups. Further examples of aliphatic groups include methyl, ethyl, propyl, butyl, isopropyl, isobutyl, vinyl, and sec-butyl. The terms “alkyl” and the prefix “alk-,” as used herein, are inclusive of both straight chain and branched saturated carbon chain. The term “alkylene,” as used herein, represents a saturated divalent straight or branched chain hydrocarbon group and is exemplified by methylene, ethylene, isopropylene and the like. The term “alkylidene,” as used herein, represents a divalent straight chain alkyl linking group. The term “alkenyl,” as used herein, represents monovalent straight or branched chain hydrocarbon group containing one or more carbon-carbon double bonds. The term “alkynyl,” as used herein, represents a monovalent straight or branched chain hydrocarbon group containing one or more carbon-carbon triple bonds.

The term “cycloaliphatic” (or “carbocycle”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule, and wherein any individual ring in said bicyclic ring system has 3-7 members. Suitable cycloaliphatic groups include, but are not limited to, cycloalkyl, cycloalkenyl, and cycloalkynyl. Further examples of aliphatic groups include cyclopentyl, cyclopentenyl, cyclohexyl, cyclohexenyl, cycloheptyl, and cycloheptenyl.

The term “heterocycle,” “heterocyclyl,” “heterocycloaliphatic,” or “heterocyclic” as used herein refers to a monocyclic, bicyclic, or tricyclic ring system in which at least one ring in the system contains one or more heteroatoms, which is the same or different, and that is completely saturated or that contains one or more units of unsaturation, but which is not aromatic, and that has a single point of attachment to the rest of the molecule. In some embodiments, the “heterocycle,” “heterocyclyl,” “heterocycloaliphatic,” or “heterocyclic” group has three to fourteen ring members in which one or more ring members is a heteroatom independently selected from oxygen, sulfur, nitrogen, or phosphorus, and each ring in the system contains 3 to 8 ring members.

Examples of heterocyclic rings include, but are not limited to, the following monocycles: 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl; and the following bicycles: 3-1H-benzimidazol-2-one, 3-(1-alkyl)-benzimidazol-2-one, indolinyl, tetrahydroquinolinyl, tetrahydroisoquinolinyl, benzothiolane, benzodithiane, and 1,3-dihydro-imidazol-2-one.

The term “heteroatom” means one or more of oxygen, sulfur, nitrogen, phosphorus, or silicon, including any oxidized form of nitrogen, sulfur, or phosphorus; the quaternized form of any basic nitrogen; or a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

The term “alkoxy,” or “thioalkyl,” as used herein, refers to an alkyl group, as previously defined, attached to the principal carbon chain through an oxygen (“alkoxy”) or sulfur (“thioalkyl”) atom.

The terms “haloalkyl,” “haloalkenyl,” and “haloalkoxy” mean alkyl, alkenyl, or alkoxy, as the case may be, substituted with one or more halogen atoms. The term “halogen” means F, Cl, Br, or I.

The term “aryl” used alone or as part of a larger moiety as in “aralkyl,” “aralkoxy,” or “aryloxyalkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 3 to 7 ring members. The term “aryl” may be used interchangeably with the term “aryl ring.” Examples of aryl rings include phenyl, naphthyl, and anthracene.

The term “heteroaryl,” used alone or as part of a larger moiety as in “heteroaralkyl,” or “heteroarylalkoxy,” refers to a monocyclic, bicyclic, and tricyclic ring system having a total of five to fourteen ring members, wherein said ring system has a single point of attachment to the rest of the molecule, at least one ring in the system is aromatic, at least one ring in the system contains one or more heteroatoms independently selected from nitrogen, oxygen, sulfur or phosphorus, and wherein each ring in the system contains 3 to 7 ring members. The term “heteroaryl” may be used interchangeably with the term “heteroaryl ring” or the term “heteroaromatic.”

Further examples of heteroaryl rings include the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

In some embodiments, an aryl (including aralkyl, aralkoxy, aryloxyalkyl, and the like) or heteroaryl (including heteroaralkyl, heteroarylalkoxy, and the like) group may contain one or more substituents. Suitable substituents on the unsaturated carbon atom of an aryl or heteroaryl group include: halogen; —R^{o}; —OR^{o}; —SR^{o}; 1,2-methylenedioxy; 1,2-ethylenedioxy; phenyl (Ph), optionally substituted with R^{o}; —O(Ph), optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(S)R^{o}; —NR^{o}C(O)N(R^{o})₂; —NR^{o}C(S)N(R^{o})₂; —NR^{o}C(O)OR^{o}; —NR^{o}NR^{o}C(O)R^{o}; —NR^{o}NR^{o}C(O)N(R^{o})₂; —NR^{o}NR^{o}C(O)OR^{o}; —C(O)C(O)R^{o}; —C(O)CH₂C(O)R^{o}; —C(O)OR^{o}; —C(O)R^{o}; —C(S)R^{o}; —C(O)N(R^{o})₂; —C(S)N(R^{o})₂; —B(OR^{o})₂; —OC(O)N(R^{o})₂; —OC(O)R^{o}; —C(O)N(OR^{o})R^{o}; —C(NOR^{o})R^{o}; —S(O)₂R^{o}; —S(O)₃R^{o}; —S(O)₂N(R^{o})₂; —S(O)R^{o}; —NR^{o}S(O)₂N(R^{o})₂; —NR^{o}S(O)₂R^{o}; —N(OR^{o})R^{o}; —C(═NH)—N(R^{o})₂; —(CH₂)₀₋₂NHC(O)R^{o}; -L-R^{o}; -L-N(R^{o})₂; -L-SR^{o}; -L-OR^{o}; -L-(C₃₋₁₀ cycloaliphatic), -L-(C₆₋₁₀ aryl), -L-(5-10 membered heteroaryl), -L-(5-10 membered heterocyclyl), oxo, C₁₋₄ haloalkoxy, C₁₋₄ haloalkyl, -L-NO₂, -L-CN, -L-OH, -L-CF₃; or two substituents, on the same carbon or on different carbons, together with the carbon or intervening carbons to which they are bound, form a 5-7 membered saturated, unsaturated, or partially saturated ring, wherein L is a C₁₋₆ alkylene group in which up to three methylene units are replaced by —NH—, —NR^{o}—, —O—, —S—, —C(O)O—, —OC(O)—, —C(O)CO—, —C(O)—, —C(O)NH—, —C(O)NR^{o}—, —C(═N—CN), —NHCO—, —NR^{o}CO—, —NHC(O)O—, —NR^{o}C(O)O—, —S(O)₂NH—, —S(O)₂NR^{o}—, —NHS(O)₂—, —NR^{o}S(O)₂—, —NHC(O)NH—, —NR^{o}C(O)NH—, —NHC(O)NR^{o}—, —NR^{o}C(O)NR^{o}, —OC(O)NH—, —OC(O)NR^{o}—, —NHS(O)₂NH—, —NR^{o}S(O)₂NH—, —NHS(O)₂NR^{o}—, —NR^{o}S(O)₂NR^{o}—, —S(O)—, or —S(O)₂—, and wherein each occurrence of R^{o} is independently selected from hydrogen, optionally substituted C₁₋₆ aliphatic, an unsubstituted 5 to 6 membered heteroaryl or heterocyclic ring, phenyl, or —CH₂(Ph), or, two independent occurrences of R^{o}, on the same substituent or different substituents, taken together with the atom(s) to which each R^{o} group is bound, form a 5-8-membered heterocyclyl, aryl, or heteroaryl ring or a 3- to 8-membered cycloalkyl ring, wherein said heteroaryl or heterocyclyl ring has 1 to 3 heteroatoms independently selected from nitrogen, oxygen, or sulfur. Non-limiting optional substituents on the aliphatic group of R^{o} include —NH₂, —NH(C₁₋₄ aliphatic), —N(C₁₋₄ aliphatic)₂, halogen, C₁₋₄ aliphatic, —OH, —O(C₁₋₄ aliphatic), —NO₂, —CN, —C(O)OH, —C(O)O(C₁₋₄ aliphatic), —O(haloC₁₋₄ aliphatic), or haloC₁₋₄ aliphatic, wherein each of the foregoing C₁₋₄ aliphatic groups of R^{o} is unsubstituted.

In some embodiments, an aliphatic or heteroaliphatic group, or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHC(O)O(alkyl), ═NNHS(O)₂(alkyl), or ═NR*, where each R* is independently selected from hydrogen or an optionally substituted C₁₋₈ aliphatic. Optional substituents on the aliphatic group of R* are selected from —NH₂, —NH(C₁₋₄ aliphatic), —N(C₁₋₄ aliphatic)₂, halogen, C₁₋₄ aliphatic, —OH, —O(C₁₋₄ aliphatic), —NO₂, —CN, —C(O)OH, —C(O)O(C₁₋₄ aliphatic), —C(O)NH₂, —C(O)NH(C₁₋₄ aliphatic), —C(O)N(C₁₋₄ aliphatic)₂, —O(halo-C₁₋₄ aliphatic), and halo(C₁₋₄ aliphatic), where each of the foregoing C₁₋₄ aliphatic groups of R* is unsubstituted; or two R* on the same nitrogen are taken together with the nitrogen to form a 5-8 membered heterocyclyl or heteroaryl ring having 1-3 heteroatoms independently selected from nitrogen, oxygen, and sulfur.

In some embodiments, optional substituents on the nitrogen of a non-aromatic heterocyclic ring include —R⁺, —N(R⁺)₂, —C(O)R⁺, —C(O)OR⁺, —C(O)C(O)R⁺, —C(O)CH₂C(O)R⁺, —S(O)₂R⁺, —S(O)₂N(R⁺)₂, —C(═S)N(R⁺)₂, —C(═NH)—N(R⁺)₂, or —NR⁺S(O)₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, aryl, or heteroaryl ring or a 3-8 membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺ are selected from —NH₂, —NH(C₁₋₄ aliphatic), —N(C₁₋₄ aliphatic)₂, halogen, C₁₋₄ aliphatic, —OH, —O(C₁₋₄ aliphatic), —NO₂, —CN, —C(O)OH, —C(O)O(C₁₋₄ aliphatic), —O(halo(C₁₋₄ aliphatic)), or halo(C₁₋₄ aliphatic), wherein each of the foregoing C₁₋₄aliphatic groups of R⁺ is unsubstituted.

As detailed above, in some embodiments, two independent occurrences of R^{o} (or R⁺, or any other variable similarly defined herein), may be taken together with the atom(s) to which each variable is bound to form a 5-8-membered heterocyclyl, aryl, or heteroaryl ring or a 3-8-membered cycloalkyl ring. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o}

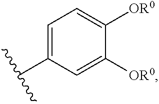

these two occurrences of R^{o} are taken together with the oxygen atoms to which they are bound to form a fused 6-membered oxygen containing ring:

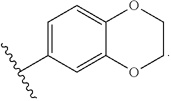

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

In some embodiments, a methylene unit of the alkyl or aliphatic chain is optionally replaced with another atom or group. Examples of such atoms or groups would include, but are not limited to, —NR^{o}—, —O—, —S—, —C(O)O—, —OC(O)—, —C(O)CO—, —C(O)—, —C(O)NR^{o}—, —C(═N—CN), —NR^{o}CO—, —NR^{o}C(O)O—, —S(O)₂NR^{o}—, —NR^{o}S(O)₂—, —NR^{o}C(O)NR^{o}—, —OC(O)NR^{o}—, —NR^{o}S(O)₂NR^{o}—, —S(O)—, or —S(O)₂—, wherein R^{o} is defined herein. Unless otherwise specified, the optional replacements form a chemically stable compound. Optional atom or group replacements can occur both within the chain and at either end of the chain; i.e. both at the point of attachment and/or also at the terminal end. Two optional replacements can also be adjacent to each other within a chain so long as it results in a chemically stable compound. Unless otherwise specified, if the replacement occurs at the terminal end, the replacement atom is bound to an H on the terminal end. For example, if one methylene unit of —CH₂CH₂CH₃ was optionally replaced with —O—, the resulting compound could be —OCH₂CH₃, —CH₂OCH₃, or —CH₂CH₂OH.

As described herein, a bond drawn from a substituent to the center of one ring within a multiple-ring system (as shown below) represents substitution of the substituent at any substitutable position in any of the rings within the multiple ring system. For example, Structure a represents possible substitution in any of the positions shown in Structure b.

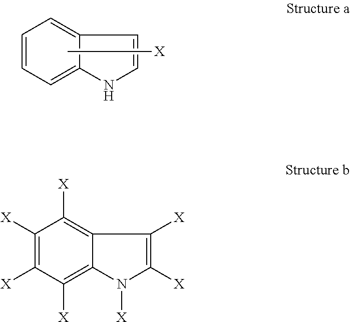

This also applies to multiple ring systems fused to optional ring systems (which would be represented by dotted lines). For example, in Structure c, X is an optional substituent both for ring A and ring B.

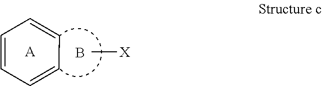

If, however, two rings in a multiple ring system each have different substituents drawn from the center of each ring, then, unless otherwise specified, each substituent only represents substitution on the ring to which it is attached. For example, in Structure d, Y is an optionally substituent for ring A only, and X is an optional substituent for ring B only.

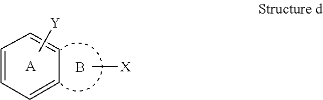

The term “protecting group,” as used herein, represent those groups intended to protect a functional group, such as, for example, an alcohol, amine, carboxyl, carbonyl, etc., against undesirable reactions during synthetic procedures. Commonly used protecting groups are disclosed in Greene and Wuts, Protective Groups In Organic Synthesis, 3^{rd} Edition (John Wiley & Sons, New York, 1999), which is incorporated herein by reference. Examples of nitrogen protecting groups include acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl and chiral auxiliaries such as protected or unprotected D, L or D, L-amino acids such as alanine, leucine, phenylalanine and the like; sulfonyl groups such as benzenesulfonyl, p-toluenesulfonyl and the like; carbamate groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl and the like, arylalkyl groups such as benzyl, triphenylmethyl, benzyloxymethyl and the like and silyl groups such as trimethylsilyl and the like. Preferred N-protecting groups are formyl, acetyl, benzoyl, pivaloyl, t-butylacetyl, alanyl, phenylsulfonyl, benzyl, t-butyloxycarbonyl (Boc) and benzyloxycarbonyl (Cbz).

The term “prodrug,” as used herein, represents a compound that is transformed in vivo into a compound of formula I or a compound listed in Table 1. Such a transformation can be affected, for example, by hydrolysis in blood or enzymatic transformation of the prodrug form to the parent form in blood or tissue. Prodrugs of the compounds of the invention may be, for example, esters. Esters that may be utilized as prodrugs in the present invention are phenyl esters, aliphatic (C₁-C₂₄) esters, acyloxymethyl esters, carbonates, carbamates, and amino acid esters. For example, a compound of the invention that contains an OH group may be acylated at this position in its prodrug form. Other prodrug forms include phosphates, such as, for example those phosphates resulting from the phosphonation of an OH group on the parent compound. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools, probes in biological assays, or as PI3K inhibitors with improved therapeutic profile.

### Description of Compounds of the Invention

In one aspect, the invention features compounds having formula I:

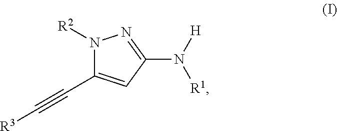

or a pharmaceutically acceptable salt thereof, wherein:
R¹ is selected from —C(O)R^{1a}, —C(O)OR^{1a}, or —C(O)N(R^{1a})(R^{1b}), wherein
R^{1a} is C₁₋₄ aliphatic, C₃₋₆ cycloaliphatic, or C₅₋₁₀ heterocyclic having up to 2 atoms selected from oxygen, sulfur, or nitrogen, wherein R^{1a} is optionally substituted with 1, 2, 3, or 4, occurrences of J^{R};
each J^{R} is independently fluoro, oxo, —C(O)J^{R1}, —C(O)N(J^{R1})₂, —C(O)O(J^{R1}), —N(J^{R1})C(O)J^{R1}, —OJ^{R1}, —SJ^{R1}, —S(O)J^{R1}, phenyl or a 5-10 membered heteroaryl or heterocyclyl ring having up to 2 atoms selected from nitrogen, oxygen, or sulfur, wherein said phenyl, heteroaryl, or heterocyclyl is optionally substituted with 1 or 2 J^{R2} groups;
each R^{1b} is, independently, hydrogen, C₁₋₄aliphatic, C₃₋₆cycloaliphatic; or
R^{1a} and R^{1b}, together with the nitrogen to which they are attached, form a 4-6 membered heterocyclic ring, wherein said heterocyclic ring optionally comprises one additional heteroatom selected from nitrogen and oxygen, and wherein said heterocyclic ring is optionally substituted with 1 or 2 J^{R2} groups;
R² is C₁₋₄aliphatic optionally substituted with 1, 2, or 3 J^{R2} groups;
each J^{R1} is independently selected from hydrogen, C₁₋₄aliphatic, C₃₋₆cycloaliphatic, phenyl, benzyl, wherein each of said C₁₋₄aliphatic, phenyl, or benzyl is optionally substituted with up to three J^{R2} groups;
each J^{R2} is, independently, selected from chloro, fluoro, —CN, —NO₂, oxo, C₁₋₄alkyl, C₃₋₆cycloaliphatic, —OH, —OC₁₋₄alkyl, —OPhenyl, or —OCH₂Phenyl; and
R³ is a 6- or 10-membered aryl ring, a 5-10-membered heterocyclic ring having up to 2 atoms selected from nitrogen, oxygen, or sulfur, or a 5-10 membered heteroaryl ring having up to 5 atoms selected from nitrogen, oxygen, or sulfur, each ring optionally substituted with up to 3 substituents independently selected from fluoro, chloro, —CN, C₁₋₄aliphatic, C₃₋₄cycloaliphatic, —OC₁₋₄aliphatic, —OC₃₋₄cycloaliphatic, or N(J^{R1})₂, wherein each of said C₁₋₄aliphatic, C₃₋₄cycloaliphatic, —OC₁₋₄aliphatic, or —OC₃₋₄cycloaliphatic is optionally substituted with up to 3 occurrences of fluoro.

In one embodiment, R¹ is selected from —C(O)R^{1a} or —C(O)NH(R^{1a}); R^{1a} is C₁₋₄ aliphatic optionally substituted with 1 or 2 occurrences of J^{R}; each J^{R} is independently fluoro, —OJ^{R1}, or a 5-membered heteroaryl ring having up to 2 atoms selected from nitrogen and optionally substituted with up to 3 J^{R2} groups; each J^{R1} is independently selected from hydrogen, C₁₋₄aliphatic, or C₃₋₆cycloaliphatic, and optionally substituted with up to three J^{R2} groups; each J^{R2} is, independently, selected from fluoro, C₁₋₄alkyl, or C₃₋₆cycloaliphatic; R² is C₁₋₄aliphatic; and R³ is a 6- or 10-membered aryl ring, having up to 2 atoms selected from nitrogen, and optionally substituted with up to 2 substituents independently selected from fluoro, chloro, C₁₋₄aliphatic, —OC₁₋₄aliphatic, or N(J^{R1})₂, wherein each of said C₁₋₄aliphatic or —OC₁₋₄aliphatic optionally substituted with up to 3 occurrences of fluoro.

In a further embodiment embodiment, R¹ is —C(O)NH(R^{1a}); R² is CH₃; and R³ is a quinolinyl, quinoxalinyl, or pyridinyl ring, each optionally substituted with up to 2 substituents independently selected from fluoro, chloro, C₁₋₄aliphatic, —OC₁₋₄aliphatic, or N(J^{R1})₂, wherein each of said C₁₋₄aliphatic or —OC₁₋₄aliphatic is optionally substituted with up to 3 occurrences of fluoro.

In yet another embodiment, R³ is an optionally substituted group selected from

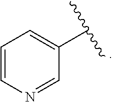

In one embodiment for a compound of formula I, R³ is optionally substituted with 1 to 2 groups independently selected from —OCH₃, Cl, F, or CF₃.

In another embodiment, R^{1a} is C₂₋₃ alkyl substituted with —OCH₃, —OCH₂CH₃, —OCH₂CH₂CH₃, —CF₃, or

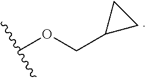

In yet another embodiment, R^{1a} is C₂₋₃ alkyl substituted with

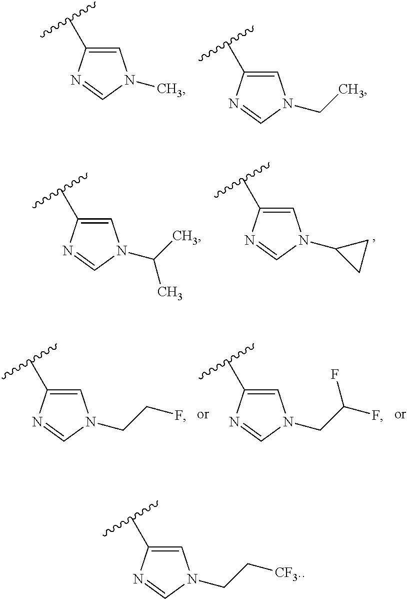

In a further embodiment, R^{1a} is C₂₋₃ alkyl, optionally substituted with one J^{R}.

In another embodiment, R^{1a} is C₂₋₃ alkyl substituted with

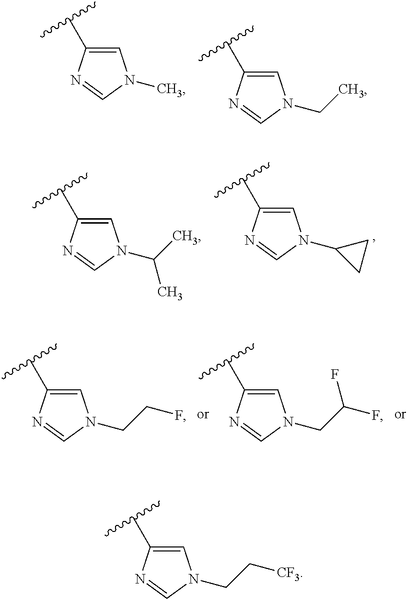

In yet another embodiment, —C(O)N(R^{1a})(R^{1b}) is

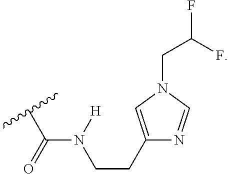

In another embodiment, the invention features a compound selected from the group of compounds listed in Table 1.

TABLE 1
1

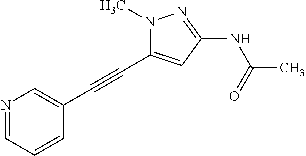

2

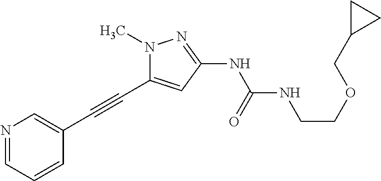

3

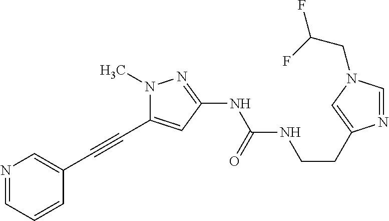

4

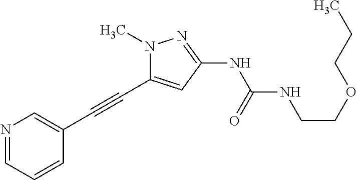

5

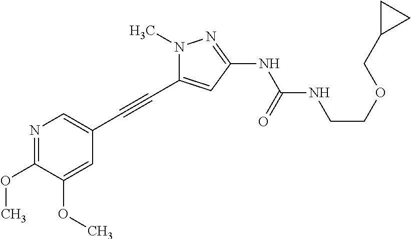

6

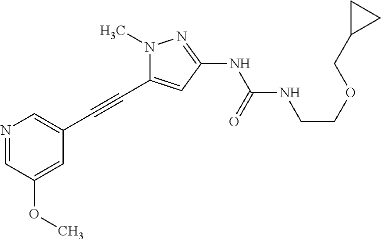

7

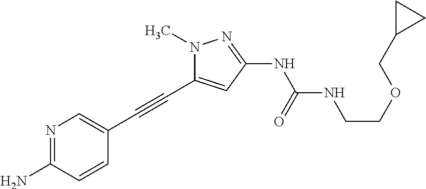

8

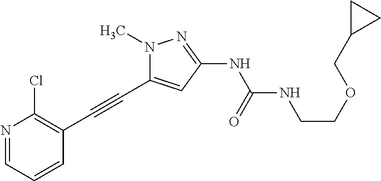

9

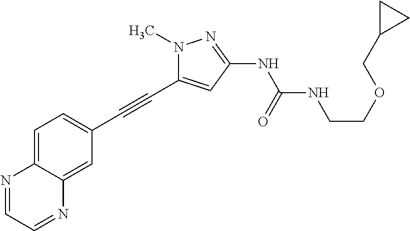

10

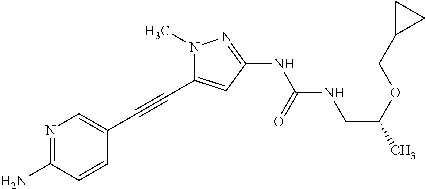

11

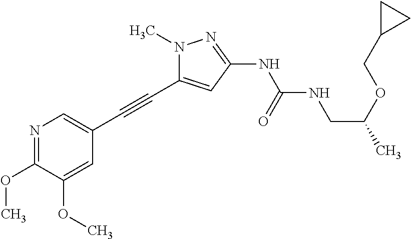

12

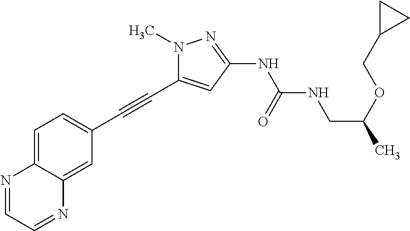

13

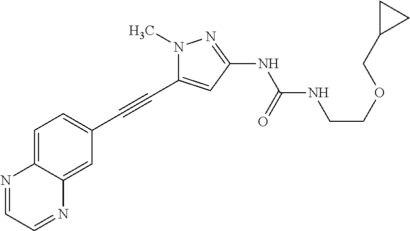

14

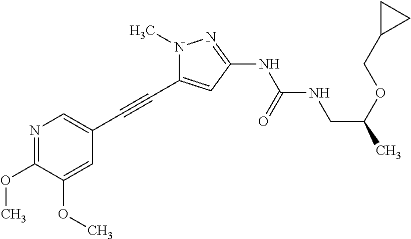

15

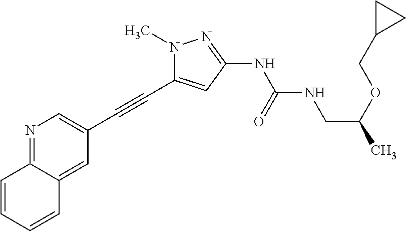

The invention also features a pharmaceutical composition comprising a compound of the invention and a pharmaceutically acceptable carrier, adjuvant, or vehicle.

In one embodiment, the composition includes a therapeutic agent selected from an agent for treating multiple sclerosis, an anti-inflammatory agent, an immunomodulatory agent, or an immunosuppressive agent.

In another embodiment, the invention features a method of treating or lessening the severity of a disease or condition selected from an autoimmune disease or an inflammatory disease of the brain or spinal cord, comprising the step of administering to said patient a compound of the invention or a pharmaceutical composition thereof.

In a further embodiment, the disease or disorder is multiple sclerosis.

In another embodiment, the method of treatment includes administering to a patient a compound or composition of the invention and an additional therapeutic agent, wherein the additional therapeutic agent is appropriate for the disease being treated and is administered together with the compound or composition as a single dosage form, or separately as part of a multiple dosage form. Examples of such additional therapeutic agents are those useful for treating multiple sclerosis, such as beta interferon, glatiramir, natalizumab, or mitoxantrone.

The invention also features a non-therapeutic method of inhibiting PI3K-gamma kinase activity in a biological sample comprising contacting said biological sample with a compound of formula I, or a composition containing said compound.

### Compositions, Formulations, and Administration of Compounds of the Invention

In another embodiment, the invention provides a pharmaceutical composition comprising a compound of any of the formulae or classes described herein. In a further embodiment, the invention provides a pharmaceutical composition comprising a compound of Table 1. In a further embodiment, the composition additionally comprises an additional therapeutic agent.

According to another embodiment, the invention provides a composition comprising a compound of this invention or a pharmaceutically acceptable derivative thereof and a pharmaceutically acceptable carrier, adjuvant, or vehicle. In one embodiment, the amount of compound in a composition of this invention is such that is effective to measurably inhibit a PI3K, particularly PI3Kγ, in a biological sample or in a patient. In another embodiment, the amount of compound in the compositions of this invention is such that is effective to measurably inhibit PI3Kα. In one embodiment, the composition of this invention is formulated for administration to a patient in need of such composition. In a further embodiment, the composition of this invention is formulated for oral administration to a patient.

The term “patient,” as used herein, means an animal, preferably a mammal, and most preferably a human.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable prodrugs, salts, esters, salts of such esters, or any other adduct or derivative which upon administration to a patient in need is capable of providing, directly or indirectly, a compound as otherwise described herein, or a metabolite or residue thereof. As used herein, the term “inhibitory active metabolite or residue thereof” means that a metabolite or residue thereof is also an inhibitor of PI3K.

As used herein, the term “pharmaceutically acceptable salt” refers to those salts which are, within the scope of sound medical judgment, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 66:1-19, 1977, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, C₁₋₈ sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. In Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, are disclosed various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intraocular, intrahepatic, intralesional, epidural, intraspinal, and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, dissolving or suspending the compound in an oil vehicle accomplishes delayed absorption of a parenterally administered compound form. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.” Examples of additional therapeutic agents are provided infra.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

### Uses of the Compounds and Compositions of the Invention

In one embodiment, the invention provides a method of inhibiting PI3K activity in the brain or spinal cord of a patient, the method comprising administering to said patient a compound or composition of the invention.

In another embodiment, the invention comprises a method of treating or lessening the severity of a PI3K-mediated condition or disease in the brain or spinal cord of a patient. The term “PI3K-mediated disease”, as used herein means any disease or other deleterious condition in which a PI3K isoform is known to play a role. In one embodiment, the PI3K isoform is PI3Kγ. In another embodiment, the PI3K isoform is PI3Kα. In a further embodiment, the invention comprises a method of treating a PI3K-mediated disease of the central nervous system. Such conditions include, without limitation, inflammatory diseases, cancer, and autoimmune-related diseases of the central nervous system. Accordingly, the invention provides a method of treating or lessening the severity of a disease of condition selected from a cancer, an autoimmune disease, or an inflammatory disease of the central nervous system of a patient, comprising administering to said patient a compound or composition of the invention.

In one embodiment, the invention provides a method of treating or lessening the severity of cancers of the brain and spinal cord. Examples of such cancers include, without limitation, high-grade invasive astrocytomas (e.g. anaplastic astrocytoma, gliobastoma multiforme), high-grade invasive astrocytomas, oligodendrogliomas, ependymomas, brain metastases, carcinomatous/lymphomatous meningitis, primary CNS lymphoma, and metastatic spinal tumors.

In another embodiment, the invention provides a method of treating or lessening the severity of an inflammatory or autoimmune disease or disorder of the central nervous system. In another embodiment, the invention provides a method of treating or lessening the severity of a symptom of an inflammatory or autoimmune disease or disorder of the central nervous system. In a further embodiment, the invention provides a method of treating neuroinflammation. Such diseases or disorders include, without limitation, multiple sclerosis, transverse myelitis, progressive multifocal leukoencephalopathy, meningitis, encephalitis, myelitis, encephalomyelitis, intracranial or intraspinal abscess, phlebitis or thrombophlebitis of intracranial venous sinuses, stroke, Parkinson's Disease, Alzheimer's Disease, Huntington's Disease, Pick's Disease, amyotrophic lateral sclerosis, HIV type-I dementia, frontotemporal lobe dementia, traumatic brain or spinal cord injury, autism, or a prion disease.

Compounds or compositions of the invention may be administered with one or more additional therapeutic agents, wherein the additional therapeutic agent is appropriate for the disease being treated and the additional therapeutic agent is administered together with a compound or composition of the invention as a single dosage form or separately from the compound or composition as part of a multiple dosage form. The additional therapeutic agent may be administered at the same time as a compound of the invention or at a different time. In the latter case, administration may be staggered by, for example, 6 hours, 12 hours, 1 day, 2 days, 3 days, 1 week, 2 weeks, 3 weeks, 1 month, or 2 months.

Non-limiting examples of chemotherapeutic agents or other anti-proliferative agents that may be combined with the compounds of this invention include taxanes, aromatase inhibitors, anthracyclines, microtubule targeting drugs, topoisomerase poison drugs, targeted monoclonal or polyconal antibodies, inhibitors of a molecular target or enzyme (e.g., a kinase inhibitor), or cytidine analogues. In one embodiment, the additional chemotherapeutic agent is amsacrine, anastrozole, asparaginase, Avastin™ (bevacizumab) azathioprine, bicalutamide, bleomycin, camptothecin, carmustine, chlorambucil, cyclophosphamide, cytarabine (araC), daunonibicin, dactinomycin, doxorubicin (adriamycin), epirubicin, epothilone, etoposide, exemestane, fludarabine, 5-fluorouracil (5-FU), flutamide, Gemzar™ (gemcitabine), Gleevec™ (imatanib), Herceptin™ (trastuzumab), idarubicin, ifosfamide, an interferon, an interleukin, irinotecan, letrozole, leuprolide, lomustine, lovastatin, mechlorethamine, megestrol, melphalan, 6-mercaptopurine, methotrexate (MTX), minosine, mitomycin, mitoxantrone, navelbine, nocodazole, platinum derivatives such as cisplatin, carboplatin and oxaliplatin, raloxifene, tamoxifen, Taxotere™ (docetaxel), Taxol™ (paclitaxel), teniposide, topotecan, tumor necrosis factor (TNF), vinblastin, vincristin, vindesine, vinorelbine, or Zoladex™ (goserelin). Another chemotherapeutic agent can also be a cytokine such as G-CSF (granulocyte colony stimulating factor). In yet another embodiment, a compound of the present invention, or a pharmaceutically acceptable salt, prodrug, metabolite, analog or derivative thereof, may be administered in combination with surgery, radiation therapy, or with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), or CMFP (cyclophosphamide, methotrexate, 5-fluorouracil and prednisone).

Additional therapeutic agents also include those useful for treating multiple sclerosis (MS), such as, for example, beta interferon (e.g., Avonex® and Rebif®), glatiramir (Copaxone®), Tysabri® (natalizumab), Betaseron® (IFN-beta), and mitoxantrone.

The invention provides a method of inhibiting PI3K kinase activity in a biological sample that includes contacting the biological sample with a compound or composition of the invention. The term “biological sample,” as used herein, means a sample outside a living organism and includes, without limitation, cell cultures or extracts thereof; biopsied material obtained from a mammal or extracts thereof; and blood, saliva, urine, feces, semen, tears, or other body fluids or extracts thereof. Inhibition of kinase activity, particularly PI3K kinase activity, in a biological sample is useful for a variety of purposes known to one of skill in the art. Examples of such purposes include, but are not limited to, biological specimen storage and biological assays. In one embodiment, the method of inhibiting PI3K kinase activity in a biological sample is limited to non-therapeutic methods.

### Preparation of Compounds of the Invention

As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997. The following definitions describe terms and abbreviations used herein:

ATP adenosine triphosphate
Brine a saturated NaCl solution in water
DCM dichloromethane
DIEA diisopropylethylamine
DMA dimethylacetamide
DMF dimethylformamide
DMSO methylsulfoxide
DTT dithiothreitol
ESMS electrospray mass spectrometry
Et₂O ethyl ether
EtOAc ethyl acetate
EtOH ethyl alcohol
HEPES 4-(2-hydroxyethyl)-1-piperazineethanesulfonic acid
HPLC high performance liquid chromatography
LC-MS liquid chromatography-mass spectrometry
Me methyl
MeOH methanol
MTBE methyl t-butyl ether
MC methyl cellulose

### NMP N-methylpyrrolidine

PBS phosphate buffered saline
Ph phenyl
RT or rt room temperature
tBu tertiary butyl
TCA trichloroacetic acid
THF tetrahydrofuran
TEA triethylamine

### General Synthetic Procedures

In general, the compounds of this invention may be prepared by methods described herein or by other methods known to those skilled in the art.

### Example 1

### General Preparation of the Compounds of Formula I

The preparation of compounds of formula I, wherein R¹ is —C(O)R^{1a}, —C(O)OR^{1a}, or —C(O)N(R^{1a})₂, is shown in Scheme 1. Accordingly, a compound of formula A1, where R² is as defined for a compound of formula I, is reacted with n-BuLi to metallate the 5-position of the pyrazole, followed by reaction with cyanogen bromide to prepare a compound of formula A2, where a bromine is at the pyrazole 5-position. The 2,5-dimethyl-1H-pyrrol-1-yl protecting group was removed by treating a compound of formula A2 with hydroxylamine to form a compound of formula A3. The primary amine of a compound of formula A3 can be reacted with activated carboxylic acids (L is an activated hydroxyl group or a halide) to form amides, such as compounds of formulae A4, wherein R^{1a} is as defined for a compound of formula I. Alternatively, the primary amine of a compound of formula A3 can be reacted with carbonyl imidazole under basic conditions to form a compound of formula A6. Subsequent reaction with amines [e.g., HN(R^{1a})(R^{1b})] or alcohols (e.g., HOR^{1a}) under basic conditions provide a compound of formula A7. Bromo pyrazoles of formulae A4 or A7 can be reacted with an optionally substituted aryl or heteroaryl acetylene (e.g., R is phenyl/napthal or 5-10 membered heteroaryl) under Sonogashira coupling conditions (Pd(PPh₃)₄/CuI/triethylamine/acetonitrile) to produce compounds of formula A5 or A8, respectively. See Chinchilla et al., Chemical Reviews 107(3): 874, 2007 for a review of the Sonogashira coupling. An exemplary preparation of 1-(2-(cyclopropylmethoxy)ethyl)-3-(5-((5,6-dimethoxypyridin-3-yl)ethynyl)-1-methyl-1H-pyrazol-3-yl)urea (compound 5) is provided in Example 3. In a variation of the synthetic route presented by Scheme 1, a compound of formula A2 can be first reacted with an optionally substituted aryl or heteroaryl acetylene under Sonogashira coupling conditions, followed by removal of the 2,5-dimethyl-1H-pyrrol-1-yl protecting group and subsequent elaboration of the resulting primary amine. Such a scheme is exemplified in the synthesis of compound 1, shown in Example 4.

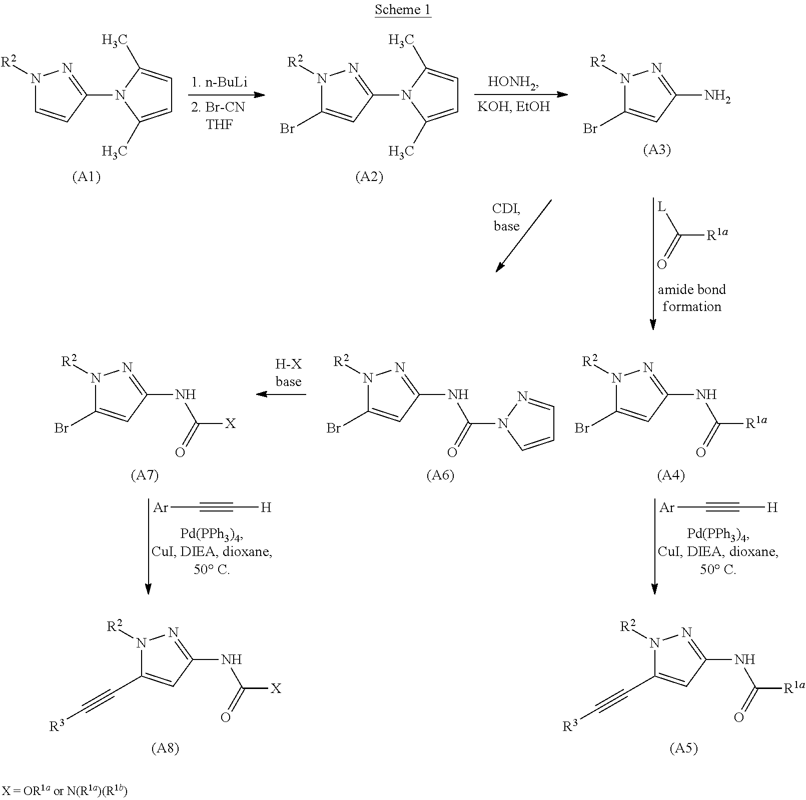

### Example 2

### Preparation of 5-ethynyl-2,3-dimethoxypyridine (Compound 1006)

As shown in step 2-i of Scheme 2, to a stirred solution of 3-methoxy-2-nitropyridine (compound 1001, 50.0 g, 325.0 mmol) in ethanol (1.0 L) and H₂O (250.0 mL) was added CaCl₂ (40.0 g, 357.0 mmol). The reaction mixture was warmed to 75° C. and iron metal (46.0 g, 811.0 mmol) was added carefully portion wise over 30 minutes. The resulting reaction mixture was stirred at 75° C. for 12 h. The reaction mixture was cooled to ambient temperature and filtered through diatomaceous earth. The earth was rinsed with EtOH (2×500 mL) and the combined filtrate concentrated under reduced pressure. The residue was suspended in EtOAc/H₂O (1:1, 2.0 L), the organic layer was separated, and the aqueous layer was extracted with EtOAc (3×1.0 L). The combined organics were dried over Na₂SO₄, filtered, and concentrated under reduced pressure to afford 2-amino-3-methoxypyridine (compound 1002, 36.0 g, 89% yield): ESMS (M+1)=125; ¹H NMR (DMSO-d₆) δ 7.5 (dd, 1H), 7.0 (dd, 1H), 6.5 (dd, 1H), 5.55)br m, 2H), 3.75 (s, 3H).

As shown in step 2-ii of Scheme 2, to a stirred solution of compound 1002 (15.0 g, 120.8 mmol) in acetic acid (150.0 mL) was added bromine (6.3 mL, 120.8 mmol) dropwise over 30 minutes at ambient temperature. The resulting reaction mixture was stirred for 16 h at ambient temperature. The reaction mixture was concentrated under reduced pressure and the acetic acid removed by the addition and subsequent removal of toluene (2×100 mL) under reduced pressure. The residue was cooled to 0° C. and neutralized with sat. aqueous NaHCO₃ solution until pH=7. The reaction mixture was extracted with EtOAc (4×500 mL). The combined organic extracts were washed with brine (60 mL), dried over Na₂SO₄, filtered, and concentrated under reduced pressure. The crude product was purified by silica gel chromatography (50% EtOAc/hexanes as eluent) to afford 5-bromo-3-methoxypyridin-2-amine (compound 1003, 20.0 g, 82% yield) as a yellow color solid: ESMS (M+1)=203/205; ¹H NMR (DMSO-d₆) δ 7.55 (d, 1H), 7.14 (d, 1H), 5.91 (br m, 2H), 3.8 (s, 3H).

As shown in step 2-iii of Scheme 2, a stirred solution of compound 1003 (10.0 g, 49.3 mmol) in 48% hydrobromic acid (95.5 mL, 566.4 mmol) was cooled to 0° C. and bromine (25.2 g, 157.6 mmol) was added, followed by the addition of 40 wt % solution of sodium nitrite (42.5 mL, 246.3 mmol) over 20 minutes. The reaction mixture, which turned into a dark black heterogeneous solution, was stirred for 1 hour at 0° C. The pH of the reaction mixture was adjusted to 13 using 50% aqueous NaOH solution. After allowing the mixture to warm to ambient temperature over 1 h, toluene (200.0 mL) was added. The reaction mixture was stirred for 30 minutes and allowed to stand overnight. The organic layer was separated and the aqueous layer was extracted with toluene (3×100 mL). The combined organics were washed with brine (40 mL), dried over Na₂SO₄, filtered, and concentrated under reduced pressure to afford 2,5-dibromo-3-methoxypyridine (compound 1004, 13.0 g, 94% yield, ˜95% purity) as a light brown solid: ESMS (M+1)=266/268/270; ¹H NMR (DMSO-d₆) δ 8.14 (s, 1H), 7.8 (s, 1H), 3.93 (s, 3H).

As shown in step 2-iv of Scheme 2, a solution of compound 1004 (13.0 g, 43.8 mmol) and 25 wt % NaOMe in methanol (95.0 mL, 438.3 mmol) was stirred for 3 hours at 75° C. After cooling, ethyl acetate and brine were added to the mixture. The organic phase was dried with MgSO₄, filtered, and concentrated under reduced pressure. The residue was purified by silica gel chromatography to provide 5-bromo-2,3-dimethoxypyridine (compound 1005, 9.1 g, 95% yield): ESMS (M+1)=218/220; ¹H NMR (DMSO-d₆) δ 7.8 (d, 1H), 7.46 (d, 1H), 3.81 (s, 3H), 3.86 (s, 3H).

As shown in step 2-v of Scheme 2, compound 1005 was suspended in 200 mL of dry THF along with PdCl₂(Ph₃P)₂ (1.56 g, 2.23 mmol) and Ph₃P (300 mg, 1.114 mmol). The mixture was flushed with N₂ for 10 minutes. Triethylamine (14.0 mL, 10.14 g, 0.1 mol) and trimethylsilylacetylene (11.3 mL, 7.84 g, 0.080 mmol) were added under a nitrogen atmosphere and stirring continued for 15 minutes more before the addition of Cu(I) iodide (500 mg; 2.65 mmol). The reaction mixture was stirred at ambient temperature for 4 hours, and then heated for 5 hours at 40° C. under N₂. The mixture was suction filtered through a pad of diatomaceous earth, which was washed with additional THF. The volatiles were removed under reduced pressure. The residue was dissolved in DCM, washed with water (2×), brine, and dried over Na₂SO₄. After filtration, the volatiles were removed under reduced pressure and the residue purified by silica gel chromatography (DCM) to provide 2,3-dimethoxy-5-((trimethylsilyl)ethynyl)pyridine (5.7 g) as a beige solid: ESMS (M+1)=236; ¹H NMR (CDCl₃) δ 7.89 (d, J=1.88 Hz, 1H), 7.1 (d, J=1.88 Hz, 1H), 4.02 (s, 3H), 3.89 (s, 3H), 0.12 (s, 9H). This material and one equivalent of powdered potassium carbonate was stirred in methyl-t-butylether/methanol/THF (1:1:1) overnight at ambient temperature. The reaction mixture was suction filtered through a pad of diatomaceous earth and washed with additional methanol. The filtrates were reduced to an oil under reduced pressure and the residue purified by silica gel chromatography (hexanes/DCM (1:1) to DCM) to provide compound 1006 (2.07 g, 39% yield) as an oil, which solidified upon standing: ESMS (M+1)=218; ¹H NMR (CDCl₃) δ 7.9 (d, J=1.8 Hz, 1H), 7.09 (d, J=1.8 Hz, 1H). 4.02 (s, 3H). 3.8 (s, 3H), 3.09 (s, 1H).

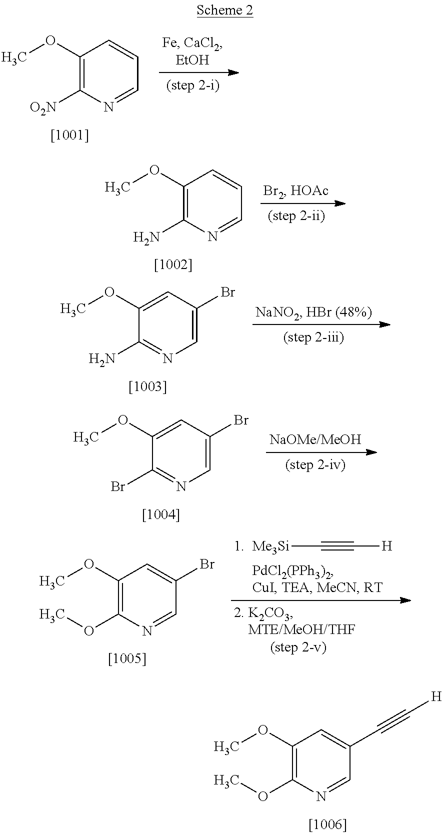

### Example 3

### Preparation of 3-N-(4-methyl-5-(2-(pyridin-3-yl)ethynyl)thiazol-2-yl)-1H-imidazole-1-carboxamide (Compound 5)

Compound 1010 was prepared according to the procedure of Chenard et al., J. Organic Chemistry, 49(7), 1124-1127, 1984. Accordingly, as shown in step 3-i of Scheme 3, 1-methyl-1H-pyrazol-3-amine (compound 1007, 2.8 g, 28.8 mmol) and 2,5-hexanedione (3.38 mL, 28.8 mmol) were dissolved into 50 mL of toluene. p-Toluenesulfonic acid (1.4 mmol) was added, the reaction mixture refluxed, and water generated from the reaction collected in a Dean-Stark trap. When no more water was generated (about 4 hours), the reaction mixture was cooled and the volatiles removed under reduced pressure. The residue was passed through a plug of silica gel using dichloromethane as eluent to yield an oil, which solidified upon standing. The solid was broken up, suspended in hexane, vigorously stirred for one hour, and collected by filtration to provide 3-(2,5-dimethyl-1H-pyrrol-1-yl)-1-methyl-1H-pyrazole (compound 1008, 5.0 g, 98% yield) as a white powder: ESMS (M+1)=175; ¹H-NMR (CDCl₃) δ 7.38 (d, J=4 Hz, 1H), 6.14 (d, J=4 Hz, 1H), 5.84 (s, 2H), 3.95 (s, 3H), 2.09 (s, 6H).

As shown in step 3-ii of Scheme 3, compound 1008 (2.0 g; 11.4 mmol) was taken up in 100 mL of dry THF under an atmosphere of nitrogen. After cooling to −78° C., 1.6M n-BuLi in hexanes (7.8 mL, 12.48 mmol) was added dropwise to the mixture. The reaction was stirred under nitrogen at −78° C. for 1.5 hours. Into a separate flask, cyanogen bromide (1.3 g, 12.4 mmol) was taken up in 3 mL of dry THF. This solution was slowly transferred to the solution of compound 1008 and the reaction allowed to come to ambient temperature. The volatiles were removed under reduced pressure and the residue was partitioned between diethyl ether and water. The organics were extracted with additional water, brine, and dried over Na₂SO₄. After filtration, the volatiles were removed under reduced pressure and the residue purified by silica gel chromatography (1:1 hexanes/DCM) to produce 5-bromo-3-(2,5-dimethyl-1H-pyrrol-1-yl)-1-methyl-1H-pyrazole (compound 1009, 1.92 g, 66.3% yield) as a white granular material: ESMS (M+1)=254; ¹H-NMR (CDCl₃) δ 6.21 (s, 1H), 5.84 (s, 2H), 3.90 (s, 3H), 2.1.0 (s, 6H).

As shown in step 3-iii of Scheme 3, hydroxylamine hydrochloride (2.6 g, 31.7 mmol) was powdered and stirred in 12 mL of ethanol at ambient temperature for 30 minutes. KOH (1.1 g, 19.52 mmol) dissolved in 1.2 mL of water and 1.2 mL of ethanol was added to the reaction mixture to form a thick white paste. A solution of compound 1009 in 4 mL of ethanol was then added and the reaction mixture heated to a gentle reflux for 23 hours. After cooling, the volatiles were removed under reduced pressure and residue partitioned between EtOAc and sat'd NaCl. A saturated solution of Na₂CO₃ was added to adjust pH to 10 and the aqueous layer extracted twice more with EtOAc. The combined organics were dried over Na₂SO₄ and the volatiles were removed under reduced pressure. The oily crystalline residue was taken up in a minimum amount of EtOAc and purified by silica gel chromatography (DCM to 5% MeOH/DCM) to provide 5-bromo-1-methyl-1H-pyrazol-3-amine (compound 1010, 1.5 g, 69.8% yield) as a white solid: ESMS (M+1)=176; ¹H-NMR (CDCl₃) δ 5.5 (s, 1H), 4.73 (br s, 2H) ex, 3.94 (s, 3H).

As shown in step 3-iv of Scheme 3, compound 1010 (4.0 g, 22.7 mmol) was suspended in 100 mL of dry DCM along with carbonyl diimidazole (CDI, 3.69 g, 22.7 mmol) and reaction was refluxed under a nitrogen atmosphere for 3.0 hours. The reaction mixture was concentrated to a minimum volume under reduced pressure and an equal volume of hexanes was added to produce a sticky granular white precipitate. The solid was collected by filtration and washed with additional DCM/hexanes (1:1) to provide N-(5-bromo-1-methyl-1H-pyrazol-3-yl)-1H-imidazole-1-carboxamide (compound 1011, 1.11 g) as a fine granular ppt. This material was used directly in the subsequent reaction as is.

As shown in step 3-v of Scheme 3,2-(cyclopropylmethoxy)-2-ethylamine (473 mg, 4.11 mmol) was added to compound 1011 (1.11 g, 4.11 mmol) in 25 mL of dry THF and the resulting solution was stirred for 12 hours at ambient temperature. The volatiles were removed under reduced pressure and the residue was purified by silica gel chromatography (DCM to 5% MeOH/DCM) to provide 1-(5-bromo-1-methyl-1H-pyrazol-3-yl)-3-(2-(cyclopropylmethoxy)ethyl)urea (compound 1012, 775 mg, 59% yield): ESMS (M+1)=318; ¹H-NMR (CDCl₃) δ 7.51 (br m, 1H), 7.19 (m, 1H), 5.90 (m, 1H), 3.76 (s, 3H), 3.61 (m, 2H), 3.51 (m, 2H), 3.33 (d, 2H), 1.07 (m, 1H), 0.54 (m, 2H), 0.50 (m, 2H).

As shown in step 3-vi of Scheme 3, compound 1012 (50 mg 0.157 mmol) and 5-ethynyl-2,3-dimethoxy pyridine (compound 1006, 183 mg, 0.197 mmol) were dissolved into 2 mL of dry dioxane. The solution was flushed with nitrogen for five minutes, followed by the addition of PdP(Ph₃)₄ (36.4 mg, 0.0315 mmol) and diisopropylamine (93 μL, 67 mg, 4.22 mmol) under an atmosphere of nitrogen. After stirring for one minute, Cu(I) iodide (6.0 mg; 0.0315 mmol) was added and reaction heated for 1 hour at 50° C. While still warm, the mixture was filtered through a pad of diatomaceous earth and washed with ether. The volatiles were removed under reduced pressure, the residue dissolved in methanol, acidified with a small amount of 6N hydrochloric acid, and purified by reversed-phase HPLC (5-100% 5.0 mM HCl MeOH/H₂O). Fractions containing pure product were concentrated to remove the methanol and the concentrate lyophilized to produce 3-N-(4-methyl-5-(2-(pyridin-3-yl)ethynyl)thiazol-2-yl)-1H-imidazole-1-carboxamide (compound 5, 11.9 mg, 27% yield) as a beige solid: ESMS (M+1)=400.

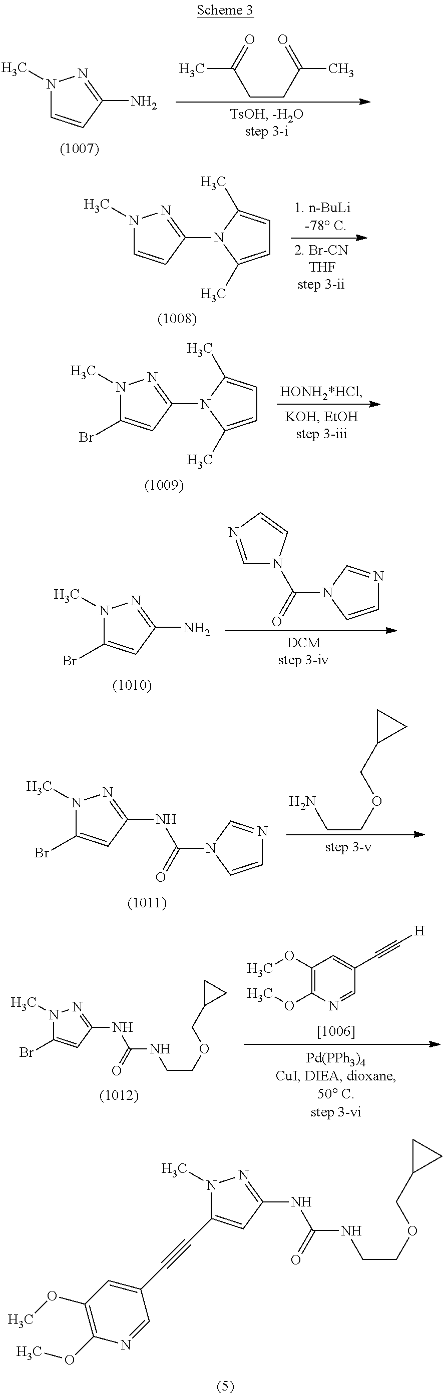

### Example 4

### Preparation of N-(1-methyl-5-(pyridin-3-ylethynyl)-1H-pyrazol-3-yl)acetamide (Compound 1)

As shown in step 4-i of Scheme 4, compound 1009 (500 mg, 1.97 mmol) and 3-ethynyl pyridine (254 mg, 2.46 mmol) were dissolved in 10 mL of dry p-dioxane and flushed with nitrogen gas for 10 minutes. Triethylamine (1.2 mL, 841 mg, 8.32 mmol) and Pd(PPh₃)₄ (254 mg, 0.394 mmol) were added and the reaction mixture flushed and purge with nitrogen gas for 3 additional minutes. Cu(I) iodide (7 4 mg, 0.394 mmol) was added and reaction was heated in an oil bath for 1 hour at 80° C. The mixture was allowed to slowly cool in the oil bath for an hour. The solids were filtered off and the filtrate was diluted with four volumes of hexanes. The resulting solution was separated from the resulting green amorphous solid and reduced to an orange oil under reduced pressure. Purification by silica gel chromatography (DCM to 2.5% MeOH/DCM) produced 3-((3-(2,5-dimethyl-1H-pyrrol-1-yl)-1-methyl-1H-pyrazol-5-yl)ethynyl)pyridine (compound 1014): ESMS (M+1)=277; ¹H-NMR (CDCl₃) δ 7.84 (m, 1H), 7.67 (m, 1H), 7.47 (m, 1H), 7.32 (m, 1H), 6.48 (s, 1H), 5.86 (s, 2H), 4.02 (s, 3H), 2.12 (s, 6H).

As shown in step 4-ii of Scheme 4, finely ground hydroxylamine hydrochloride (360 mg, 5.17 mmol) was stirred in 2.0 mL ethanol for 30 minutes, followed by the addition of KOH (179 mg, 3.18 mmol) in ethanol/water (1:1, 400 μL total volume) to this stirring suspension to form a thick white paste. Compound 1014 (550 mg, 1.99 mmol) was added as a concentrated solution in ethanol and the reaction mixture heated to 80° C. for 24 hours. The volatiles were removed under reduced pressure and the residue partitioned between water and EtOAc. The organics were washed with water, brine, and dried over Na₂SO₄. After filtration, the volatiles were removed under reduced pressure to produce 1-methyl-5-(pyridin-3-ylethynyl)-1H-pyrazol-3-amine (compound 1015) as a red oil which crystallized upon standing: ESMS (M+1)=199. This material was taken on to next step without further purification.

Compound 1015 from step 4-ii was dissolved into 20 mL of dry THF and diisopropylethylamine (411 μL, 305 mg, 2.36 mmol) was added, followed by the addition of acetyl chloride (150 μL, 165 mg, 2.08 mmol). The reaction was stirred for 30 minutes at ambient temperature and the volatiles removed under reduced pressure. The residue was triturated with Et₂O and the precipitate isolated, dissolved in a minimum amount of EtOAc and filtered through a plug of silica gel with EtOAc as eluent. The filtrates were concentrated under reduced pressure to provide N-(1-methyl-5-(pyridin-3-ylethynyl)-1H-pyrazol-3-yl)acetamide (compound 1, 400 mg after conversion to HCl salt) as a beige solid: ESMS (M+1)=241; ¹H-NMR (CDCl₃) δ 8.77 (d, J=1.8 Hz, 1H), 8.60 (dd, J=1.8 Hz, 5 Hz; 1H), 7.8 (ddd, J=1.8 Hz, 5 Hz, 8 Hz; 7.7 (br m, 1H), 7.2 (dd, J=5 Hz, 8 Hz; 1H), 3.88 (s, 3H), 2.16 (s, 3H).

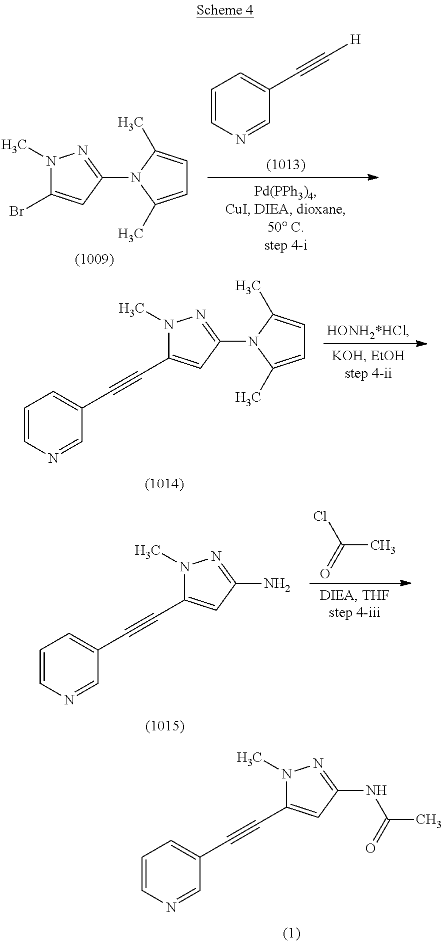

### Example 5

### Preparation of (R)-2-(cyclopropylmethoxy)propan-1-amine (compound 1019)

As shown in step 5-i of Scheme 5, sodium cyanoborohydride (5.23 g, 40.85 mL, 83.21 mmol) was added in small portions over 15 minutes to a stirred solution of (R)-aminopropan-2-ol (compound 1016, 5.00 g, 66.57 mmol) and benzaldehyde (14.12 g, 133.14 mmol) in anhydrous methanol (90 mL) and glacial acetic acid (10 mL) at room temperature. The resulting yellow solution was heated at 60° C. for 3 hours and cooled to room temperature. The volatiles were removed under reduced pressure and the residue dissolved in water (200 mL) and made basic with saturated NaHCO₃ solution (200 mL). The aqueous phase was extracted with EtOAc (3×100 mL). The combined organic layers were dried over Na₂SO₄, filtered, and the volatiles removed under reduced pressure to yield an oil, which was purified by medium pressure silica gel chromatography (elution with 0-30% EtOAc/hexanes) to provide (R)-1-(dibenzylamino)propan-2-ol (compound 1017, 12.0 g, 70.6% yield) as a clear viscous oil: ESMS (M+1)=256.26; ¹H-NMR (CDCl₃) δ 7.26-7.38 (m, 10H), 3.87 and 3.42 (ABq, J=12.0 Hz, 4H), 3.30 (m, 1H), 2.43 (d, J=9.0 Hz, 2H), 1.08 (d, J=6.0 Hz, 3H).

As shown in step 5-ii of Scheme 5, sodium hydride (1.278 g, 53.24 mmol) was added in small portions to a stirred solution of compound 1017 (3.4 g, 13.31 mmol) in anhydrous DMF (10 mL) at room temperature under nitrogen. The resulting suspension was stirred at room temperature for 30 minutes and then heated at 75° C. for 8 hours. After cooling to room temperature, the solution was poured into water (50 mL) and sat.NaHCO₃ (25 mL). The aqueous layer was extracted with ether/EtOAc (2:1, 2×100 mL), and the combined organics were dried over Na₂SO₄ and concentrated to give an oil. The crude product was purified by medium pressure silica gel chromatography (elution with 0-10% EtOAc/hexanes) to provide (R)—N,N-dibenzyl-2-(cyclopropylmethoxy)propan-1-amine (compound 1018, 3.1 g, 78%) as a clear viscous oil: ESMS (M+1)=310.32; ¹H-NMR (CDCl₃) δ 7.19-7.38 (m, 10H), 3.70 and 3.50 (ABq, J=15.0 Hz, 4H), 3.55 (m, 1H), 3.24 (m, 2H), 2.59 (dd, J=6.0, 15.0 Hz, 1H), 2.42 (dd, J=6.0, 15.0 Hz, 1H) 1.08 (d, J=6.0 Hz, 3H), 1.02 (m, 1H), 0.48 (m, 2H), 0.15 (m, 2H).

As shown in step 5-iii of Scheme 5, 10% Pd on C (934 mg, 4.39 mmol) was added to a stirred, nitrogen degassed solution containing ammonium formate (2.45 g, 38.78 mmol) and compound 1018 (2.00 g, 6.46 mmol) in methanol (50 mL) at room temperature. After addition, the reaction mixture was heated at 60° C. for 1 hr, cooled to room temperature, and filtered through diatomaceous earth, which was subsequently washed with methanol (2×25 mL). The combined filtrates were concentrated under reduced pressure. The resulting oil was suspended in dichloromethane (10 mL), dried over Na₂SO₄, filtered, and the collected solids washed with dichloromethane (10 mL). The combined filtrates were concentrated under reduced pressure to provide (R)-2-(cyclopropylmethoxy)propan-1-amine (compound 1019, 0.72 g, 86% yield) as a pale yellow oil: ¹H-NMR (CDCl₃) δ 3.21-3.45 (m, 3H), 2.67 (m, 2H) 1.09 (d, J=6.0 Hz, 3H), 1.02 (m, 1H), 0.51 (m, 2H), 0.18 (m, 2H). (S)-2-(Cyclopropylmethoxy)propan-1-amine can be similarly prepared starting with (S)-aminopropan-2-ol.

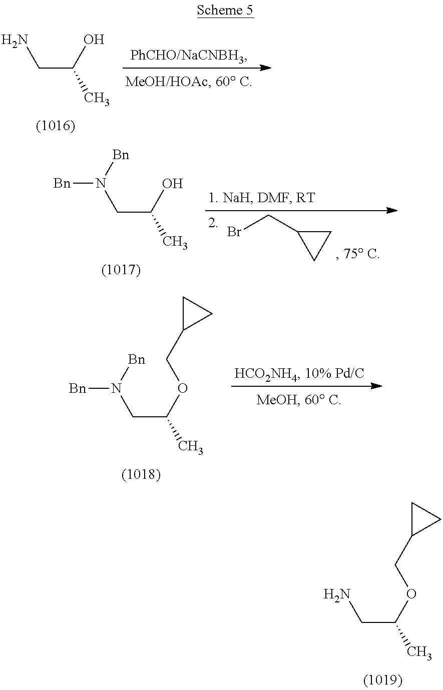

Table 2 provides analytical characterization data for certain compounds of formula I (blank cells indicate that the test was not performed). Compound numbers in Table 2 correspond to those depicted in Table 1.

TABLE 2

| Compound | ESMS | ¹H NMR (300 MHz, unless indicated otherwise) |
|---|---|---|
| No. | (M + H) | NMR peaks given as δ values |
| 1 | 241 | (CDCl₃): δ 8.75 (d, 1H), 8.60 (dd, 1H), 7.81 (dt, 1H), |
| | | 7.40 (br m, 1H), 7.2 (dd, 1H), 6.92 (s, 1H), 3.89 (s, 3H). |
| | | 2.15 (s, 3H) |
| 2 | 340 | |
| 3 | 400 | |
| 4 | 328 | |
| 5 | 400 | |
| 6 | 370 | |
| 7 | 355 | |
| 8 | 374 | |
| 9 | 391 | |
| 10 | 369 | (CDCl₃): 8.85 (s, 1H), 8.13 (d, J = 2.8:1H), 7.5 (dd, |
| | | J = 2.8 Hz, 8.25 Hz; 1H), 6.62 (br m, 1H), 6.29 (s, 1H), |
| | | 3.73 (s, 3H), 3.48 (m, 1H). 3.25 (m, 5H), 3.05 (m, H), |
| | | 1.1, d, J = 5 Hz; 3H), 1.01 (m, 1H), 0.45 (m, 2H), 0.2 (m, 2H) |
| 11 | 414 | (DMSO-d₆): 8.0 (d, J = 1/8 Hz; 1H), 7.3 (br m, 1H), 7.0 (d, |
| | | J = 1.8 Hz; 1H), 6.05 (s, 1H), 4.05 (s, 3H), 3.90 (s, 3H), |
| | | 3.86 (s, 3H). 3.5 (m, 2H), 3.6 (m, 2H), 3.34 (m, 1H), |
| | | 1.19, d, J = 5 Hz; 3H), 1.01 (m, 1H), 0.51 (m, 2H), |
| | | 0.39 (m, 2H) |
| 12 | 405 | |
| 13 | 390 | |
| 14 | 414 | |
| 15 | 404 | |

### Biological Assay of Compounds of the Invention

### Example 4

### PI3K Inhibition Assay

Using a Biomek FX from Beckman Coulter, 1.5 μL of each of ten 2.5-fold serial dilutions of a compound of the invention in 100% DMSO was added to an individual well (hereafter, “test well”) in a 96 well polystyrene plate [Corning, Costar Item No. 3697]. One test well also contained 1.5 μL of DMSO with no compound. Another well contained an inhibitor in DMSO at a concentration known to completely inhibit the enzyme, (hereafter “background well”). Using a Titertek Multidrop, 50 μL of Reaction Mix [100 mM HEPES pH 7.5, 50 mM NaCl, 10 mM DTT, 0.2 mg/mL BSA, 60 μM phosphatidylinositol(4,5)bisphosphate diC16 (PI(4,5)P₂; Avanti Polar Lipids, Cat. No. 840046P) and PI3K isoform of interest (see Table 3 for isoform concentrations)] was added to each well. To initiate the reaction, 50 μL of ATP Mix [20 mM MgCl₂, 6 μM ATP (100 μCi/μmmole ³³P-ATP)] was added each well, followed by incubating the wells for 30 min. at 25° C. Final concentrations in each well were 50 mM HEPES 7.5, 10 mM MgCl₂, 25 mM NaCl, 5 mM DTT, 0.1 mg/mL BSA, 30 μM PI(4,5)P₂, 3 μM ATP, and the PI3K isoform of interest (see Table 3). Final compound concentrations in each well ranged from 10 μM to 1 nM.

TABLE 3

| | PI3K Isoform Concentrations | | | |
|---|---|---|---|---|
| | PI3K-α | PI3K-β | PI3K-γ | PI3K-δ |
| Enzyme concentration in | 4 nM | 20 nM | 4 nM | 4 nM |
| Reaction Mix | | | | |
| Final enzyme concentration | 2 nM | 10 nM | 2 nM | 2 nM |

After incubation, the reactions in each well were quenched by addition of 50 μL of stop solution [30% TCA/Water, 10 mM ATP]. Each quenched reaction mixture was then transferred to a 96 well glass fiber filter plate [Corning, Costar Item No. 3511]. The plate was vacuum-filtered and washed three times with 150 μL of 5% TCA/water in a modified Bio-Tek Instruments ELX-405 Auto Plate Washer. 50 μL of scintillation fluid was added to each well and the plate read on a Perkin-Elmer TopCount™ NXT liquid scintillation counter to obtain ³³P-counts representing inhibition values.

The value for the background well was subtracted from the value obtained for each test well and the data were fit to the competitive tight binding Ki equation described by Morrison and Stone, Comments Mol. Cell. Biophys. 2: 347-368, 1985.

Each of compounds 1 to 15 had a Ki of less than 2 micromolar for the inhibition of PI3K-gamma. Each of compounds 3, 5-9, 11, 12, and 14 had a Ki of less than 0.10 micromolar for the inhibition of PI3K-gamma.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound having the formula: 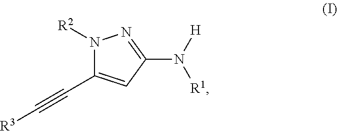
or a pharmaceutically acceptable salt thereof, wherein:
R¹ is selected from —C(O)R^{1a}, —C(O)OR^{1a}, or —C(O)N(R^{1a})(R^{1b});
R^{1a} is C₁₋₄ aliphatic, C₃₋₆ cycloaliphatic, or C₅₋₁₀ heterocyclic having up to 2 atoms selected from oxygen, sulfur, or nitrogen, wherein R^{1a} is optionally substituted with 1, 2, 3, or 4, occurrences of J^{R};
each J^{R} is independently fluoro, oxo, —C(O)J^{R1}, —C(O)N(J^{R1})₂, —C(O)O(J^{R1}), —N(J^{R1})C(O)J^{R1}, —OJ^{R1}, —SJ^{R1}, S(O)J^{R1}, phenyl or a 5-10 membered heteroaryl or heterocyclyl ring having up to 2 atoms selected from nitrogen, oxygen, or sulfur, wherein said phenyl, heteroaryl, or heterocyclyl is optionally substituted with 1 or 2 J^{R2} groups;
each R^{1b} is, independently, hydrogen, C₁₋₄aliphatic, C₃₋₆cycloaliphatic; or
R^{1a} and R^{1b}, together with the nitrogen to which they are attached, form a 4-6 membered heterocyclic ring, wherein said heterocyclic ring optionally comprises one additional heteroatom selected from nitrogen and oxygen, and wherein said heterocyclic ring is optionally substituted with 1 or 2 J^{R2} groups;
R² is C₁₋₄aliphatic optionally substituted with 1, 2, or 3 J^{R2} groups;
each J^{R1} is independently selected from hydrogen, C₁₋₄aliphatic, C₃₋₆cycloaliphatic, phenyl, benzyl, wherein each of said C₁₋₄aliphatic, phenyl, or benzyl is optionally substituted with up to three J^{R2} groups;
each J^{R2} is, independently, selected from chloro, fluoro, —CN, —NO₂, oxo, C₁₋₄alkyl, C₃₋₆cycloaliphatic, —OH, —OC₁₋₄alkyl, —OPhenyl, or —OCH₂Phenyl; and
R³ is a 6- or 10-membered aryl ring, a 5-10-membered heterocyclic ring having up to 2 atoms selected from nitrogen, oxygen, or sulfur, or a 5-10 membered heteroaryl ring having up to 5 atoms selected from nitrogen, oxygen, or sulfur, each ring optionally substituted with up to 3 substituents independently selected from fluoro, chloro, —CN, C₁₋₄aliphatic, C₃₋₄cycloaliphatic, —OC₁₋₄aliphatic, —OC₃₋₄cycloaliphatic, or N(J^{R1})₂, wherein each of said C₁₋₄aliphatic, C₃₋₄cycloaliphatic, —OC₁₋₄aliphatic, or —OC₃₋₄cycloaliphatic is optionally substituted with up to 3 occurrences of fluoro.

2. The compound according to claim 1, or a pharmaceutically acceptable salt thereof, wherein:
R¹ is selected from —C(O)R^{1a} or —C(O)NH(R^{1a});
R^{1a} is C₁₋₄ aliphatic optionally substituted with 1 or 2 occurrences of J^{R};
each J^{R} is independently fluoro, —OJ^{R1}, or a 5-membered heteroaryl ring having up to 2 atoms selected from nitrogen and optionally substituted with up to 3 J^{R2} groups;
each J^{R1} is independently selected from hydrogen, C₁₋₄aliphatic, or C₃₋₆cycloaliphatic, and optionally substituted with up to three J^{R2} groups;
each J^{R2} is, independently, selected from fluoro, C₁₋₄alkyl, or C₃₋₆cycloaliphatic;
R² is C₁₋₄aliphatic; and
R³ is a 6- or 10-membered aryl ring, having up to 2 atoms selected from nitrogen, and optionally substituted with up to 2 substituents independently selected from fluoro, chloro, C₁₋₄aliphatic, —OC₁₋₄aliphatic, or N(J^{R1})₂, wherein each of said C₁₋₄aliphatic or —OC₁₋₄aliphatic optionally substituted with up to 3 occurrences of fluoro.

3. The compound according to claim 1, or a pharmaceutically acceptable salt thereof, wherein or a pharmaceutically acceptable salt thereof, wherein:
R¹ is —C(O)NH(R^{1a});
R² is CH₃; and
R³ is a quinolinyl, quinoxalinyl, or pyridinyl ring, each optionally substituted with up to 2 substituents independently selected from fluoro, chloro, C₁₋₄aliphatic, —OC₁₋₄aliphatic, or N(J^{R1})₂, wherein each of said C₁₋₄aliphatic or —OC₁₋₄aliphatic is optionally substituted with up to 3 occurrences of fluoro.

4. The compound according to claim 2, or a pharmaceutically acceptable salt thereof, wherein R³ is an optionally substituted group selected from

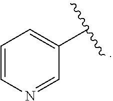

5. The compound according to claim 3, wherein R³ is optionally substituted with 1 to 2 groups independently selected from —OCH₃, Cl, F, or CF₃.

6. The compound according to claim 1, or a pharmaceutically acceptable salt thereof, wherein R^{1a} is C₂₋₃ alkyl substituted with —OCH₃, —OCH₂CH₃, —OCH₂CH₂CH₃, —CF₃, or

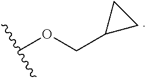

7. The compound according to claim 1, or a pharmaceutically acceptable salt thereof, wherein R^{1a} is C₂₋₃ alkyl substituted with

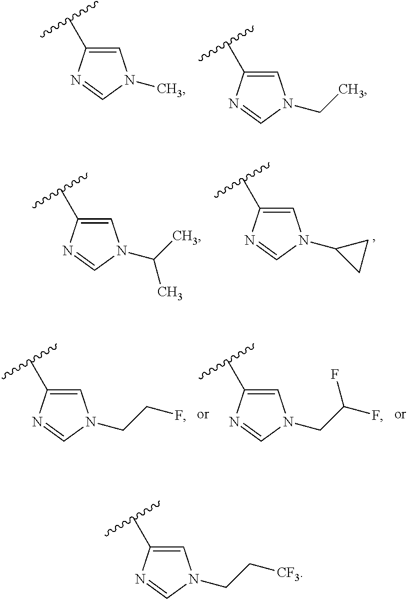

8. The compound according to claim 1, or a pharmaceutically acceptable salt thereof, wherein —C(O)N(R^{1a})(R^{1b}) is

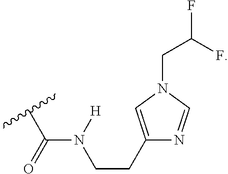

9. The compound according to claim 1, or a pharmaceutically acceptable salt thereof, wherein said compound is selected from

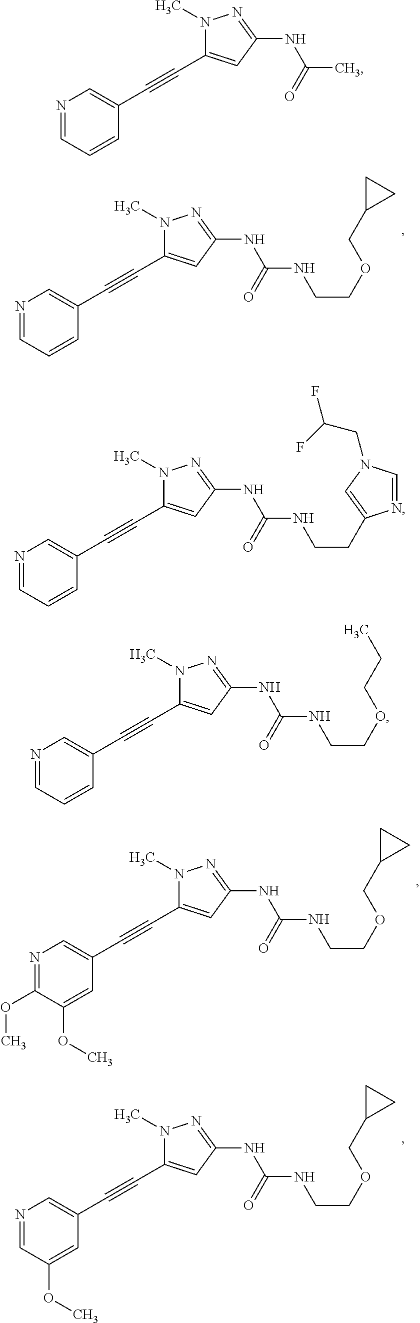

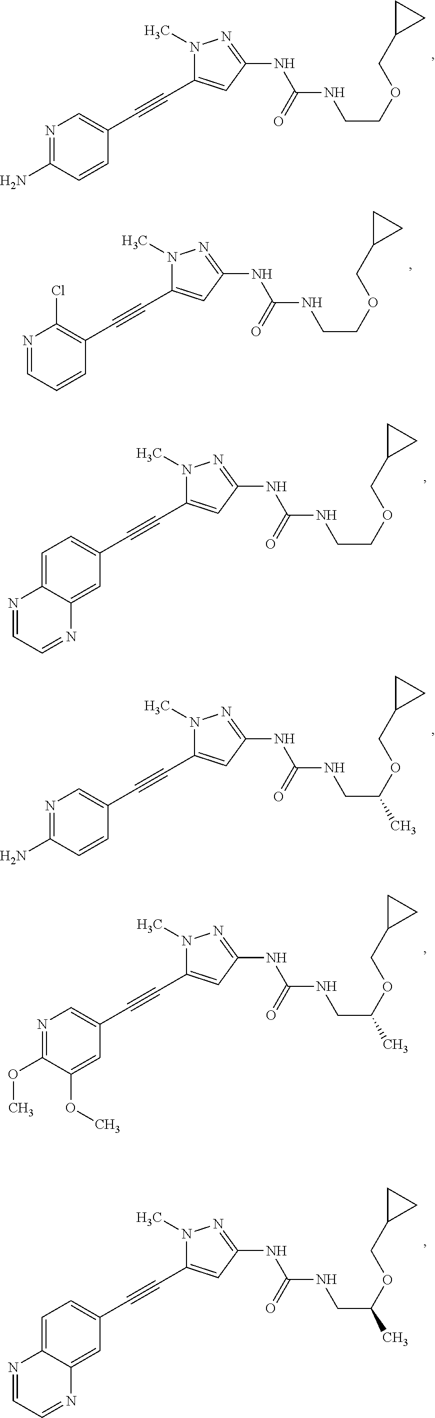

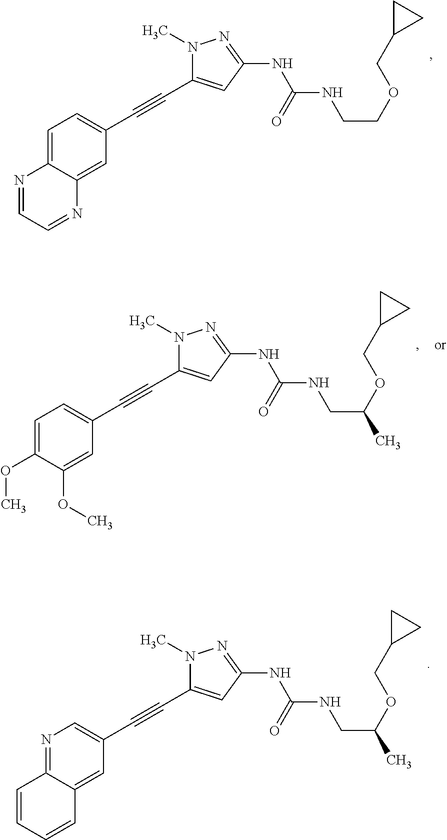

10. A pharmaceutical composition comprising a compound according to claim 1 and a pharmaceutically acceptable carrier, adjuvant, or vehicle.

11. A method of treating or lessening the severity of a disease or condition selected from an autoimmune disease or an inflammatory disease of the brain or spinal cord, comprising the step of administering to said patient a compound or salt thereof according to claim 1, or a pharmaceutical composition thereof.

12. The method according to claim 11, wherein said disease or disorder is multiple sclerosis.

13. The method according to claim 11, comprising the additional step of administering to said patient an additional therapeutic agent, wherein said additional therapeutic agent is appropriate for the disease being treated and said additional therapeutic agent is administered together with said compound or composition as a single dosage form or separately from said compound or composition as part of a multiple dosage form and is selected from beta interferon, glatiramir, natalizumab, or mitoxantrone.

14. A method of inhibiting PI3K-gamma kinase activity in a biological sample comprising contacting said biological sample with a compound according to claim 1 or a composition according to claim 10.
